(19) United States
(12) Patent Application Publication
(10) Pub. No.: US 2007/0195268 A1
(43) Pub. Date: Aug. 23, 2007

(54) ADVANCED CORNEAL TOPOGRAPHY SYSTEM

(76) Inventors: Edwin J. Sarver, Carbondale, IL (US); James Marous, South Vienna, OH (US); Cynthia Roberts, Columbus, OH (US)

Correspondence Address: BOND, SCHOENECK & KING, PLLC, 10 BROWN ROAD, SUITE 201, ITHACA, NY, 14850-1248, US

(21) Appl. No.: 11/674,985

(22) Filed: Feb. 14, 2007

Related U.S. Application Data
(60) Provisional application No. 60/773,293, filed on Feb. 14, 2006.

(51) Int. Cl. A61B 3/10

(52) U.S. Cl. 351/212

(57) ABSTRACT

The purpose of this invention is to provide a corneal topography system for use by eye car professionals to diagnose and correct vision defects; particularly, to an advanced corneal topography system which accurately assess the shape of the human cornea utilizing a projected grid and current digital camera technology.

This application relates to provisional application No. 60/773,293, filed Feb. 14, 2006. This invention relates to a corneal topography system for use by eye care professionals to diagnose and correct vision defects; particularly, to an advanced corneal topography system which accurately assess the shape of the human cornea utilizing a projected grid and current digital camera technology.

### BACKGROUND OF THE INVENTION

### FIELD OF THE INVENTION

The human cornea is the clear window of the eye. It provides about ¾ of the refractive power of the eye; thus, it is of great interest to accurately assess the eye's topography (shape). This shape information can be used to diagnose corneal disease (such as keratoconous) as well as planning for vision correction using contact lenses or corneal refractive surgery.

Currently available corneal topography systems include those based on measuring reflected concentric light rings and scanning light slits. Those based on reflected light rings suffer from the inability to measure highly aberrated corneas where a single ring can cause multiple reflections. Systems based on scanning light slits operate by imaging individual images of a bright slit of light which is diffusely reflected. This diffusely reflected image does not have edges as sharp as those which are specularly reflected by the ring systems. As a result, the inherent measurement accuracy is reduced. In addition, since the slits must be scanned over some finite time period in which the eye can be moving, there are problems in providing an exact registration of the individual images relative to the eye being measured.

During the time period in which it was commercially available, the PAR Corneal Topography System (PAR CTS) represented a unique technology for measuring corneal surface shape that was not dependent on a specular reflection, and therefore not dependent on high quality optical surfaces or strict alignment criteria. The PAR CTS was developed, manufactured, and marketed by PAR Vision Systems (PAR Vision), a wholly owned subsidiary of PAR Technology, Inc. (PAR TECH). It was an “elevation” system, distinguishing it from the market leading Placido systems, which measures surface slope rather than height. In addition, the PAR CTS required the application of topical fluorescein to acquire the base measurement, which was not necessary with a Placido device.

The PAR CTS was pulled from the market in 1998, due to a strategic decision by the parent company. However, many PAR CTS systems were sold worldwide prior to 1998 and many are still being used by clinicians who feel strongly that the unique technology provides diagnostic information that cannot be obtained with a Placido system. These clinicians prefer the PAR CTS over other topographic systems in their office for specific patients, despite the fact that the computer (pre-Pentium) and operating system (Windows 3.11) are now excruciatingly slow and cumbersome, no longer supported by the company, and were functionally obsolete.

The aforementioned grid projection system partially addressed these shortcomings but had limitations in that the image processing was marginal and required specialized hardware that is no longer available. It also had a long working distance and long optical layout that made it difficult to adapt to surgical microscopes, thus limiting its utility.

The present corneal typographical system, referred to as rasterstereography corneal topography (RCT) system, overcomes the abovementioned limitations to provide accurate corneal measurements for use by eye care professionals to diagnose and correct vision defects. The instant invention utilizes a projected grid that provides a diffuse reflection, thus, it does not suffer from double reflections which can confuse the reconstruction algorithm of concentric ring systems. The image processing of the instant invention does not require special hardware. Moreover, the entire system has been made more amenable to operate with a surgical microscope.

### SUMMARY OF THE INVENTION

The purpose of the present invention is to provide accurate corneal measurements for use by eye care professionals capable of diagnosing and correcting a patient's vision.

The present invention utilizes a projected grid that provides a diffuse reflection at the tear layer on the cornea. The tears are stained with fluorescein so that when the cyan grid is projected, the light becomes fluorescent and can be imaged by a grid camera. The entire grid is captured in a single frame. The inventive system utilizes a fast and robust image processing algorithm to extract the grid features and yield an accurate and detailed corneal surface representation.

Accordingly, it is an objective of the instant invention to provide a grid that is diffusely reflected such that it does not provide double reflections which can confuse the reconstruction algorithm of concentric ring systems.

It is yet another objective of the instant invention to teach a corneal topography system wherein the entire grid is captured in a single frame, thereby avoiding the problem of sequential image registration.

Another objective of the present invention is to provide a corneal topography system wherein the image processing has been improved and no longer requires special hardware.

Still another objective of the instant invention is to teach an optical layout having a small package design to thereby shorten the entire system making it more amenable to operation with a surgical microscope. Optical design was optimized, and the cumbersome Zenon flash system of the prior art was eliminated to take advantage of modern LED based illumination systems.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 illustrates a typical input grid image;

FIG. 2 illustrates row and column sums to find the location of the center of grid pattern;

FIG. 3 illustrates a profile of column sums in the center region of interest (ROI) for the image shown in FIG. 1;

FIG. 4 illustrates the results of center finding for the image shown in FIG. 1. The red square indicates the center ROI where the row and column sums are computed. The red dot in the center shows the computed cross center;

FIG. 5 illustrates the results of vertical linear feature enhancement via convolution operation;

FIG. 6 illustrates peak detection algorithm, where the high threshold is H and low threshold is L; the peaks are the local maxima and the valleys are the local minima;

FIG. 7 illustrates the final result of the vertical features processing after thinning, binarizing, and shot noise removal;

FIG. 8 illustrates the processing results of horizontal features processing after thinning, binarizing, and shot noise removal;

FIG. 9 illustrates a case when the linear features actually intersect but is not detected because the continuous horizontal linear feature and the continuous vertical linear feature cross at the boundary of a pixel;

FIG. 10 illustrates feature intersections. Note: in this image the spots have been greatly expanded in size for clarity, in the actual image each dot was a single pixel;

FIG. 11 illustrates the process of searching for neighboring nodes about the current node;

FIG. 12 illustrates the final results where the final nodes overlay on top of original image;

FIG. 13 shows a schematic diagram of a Zemax ray tracing of front view camera;

FIG. 14 shows a schematic diagram of a Zemax ray tracing of the grid camera;

FIG. 15 shows a schematic diagram of a Zemax ray tracing for grid projection;

FIG. 16 shows a schematic diagram of a Zemax ray trace for the grid projector;

FIG. 17a illustrates a top view of an optical assembly used in the present invention;

FIG. 17b illustrates a side view of the optical assembly of FIG. 17a;

FIG. 18 illustrates details of an optical mount used in the instant assembly;

FIG. 19 is a schematic for the voltage power supply (i.e., 5 volt) in the flash controller;

FIG. 20 is a schematic for the connectors and switches in the flash controller;

FIG. 21 is a schematic for the LED array drivers in the flash controller;

FIG. 22 is a schematic for the one shot trigger n the flash controller;

FIG. 23 is a schematic for the programmable logic device (PLD) in the flash controller;

FIG. 24 is a schematic for the programmable current power supply (i.e., 2 amp) in the flash controller.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

The goal of grid intersection extraction algorithm, outlined below, is to extract the grid features from a rasterstereography corneal topography (RCT) system captured image. Once the features have been extracted, the surface can be reconstructed. In a preferred embodiment, the total process time for the image should be less than 2.0 seconds; for example, the processing time for an algorithm running on a 3 GHz PC is about 0.2 seconds.

The primary task is to find the center of the grid and all grid intersections (called nodes) in the image. By way of an overview, the main steps of the algorithm are:

1. find the center of the grid using histograms of row and columns sums around the center of the image. This is the starting node to be used below.

2. extract horizontal and vertical line features by:

enhancing the vertical lines in the image via a filtering process and save in an array;

enhancing the horizontal lines in the image via a filtering process and save in an array;

post-processing the vertical lines array to thin the vertical lines and remove small noise features;

post-processing the horizontal lines array to thin the horizontal lines and remove small noise features; and

3. Find the intersections in the post-processed vertical and horizontal lines arrays starting with the center of the grid, find all horizontal and vertical (4-connected) neighbors of a node and add all new nodes to a stack. (Each new intersection will have up to 3 neighbors yet to be found);

if the stack is not empty, pop a node off the stack and find its neighbors.

4. Continue adding new nodes to the stack and processing nodes until the stack is empty.

A feature of the algorithm not described in the basic steps outlined above is the ability to use prediction to estimate where neighboring nodes are expected to be located. By iterating over the set of found nodes and gradually relaxing processing thresholds, the hard to locate node locations (for example in areas of low contrast) can be reliably extracted.

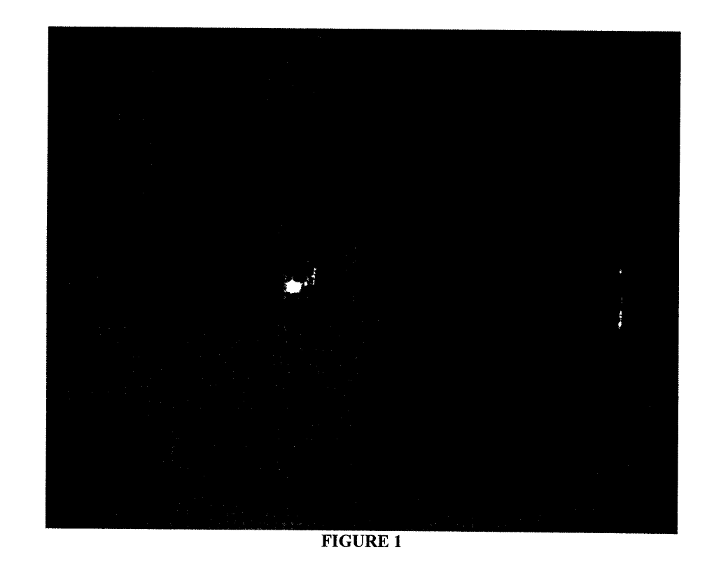

The specific algorithm details of these steps are discussed below with respect to FIG. 1, which shows a typical input image.

The first step in the image processing algorithm is to detect the center of the bright cross near the center of the image.

Center Finding

As observed in FIG. 1, the center cross feature is brighter than surrounding pixels. Also the horizontal and vertical sections are generally aligned with rows and columns of the image. These characteristics are exploited by the present invention to locate the center cross.

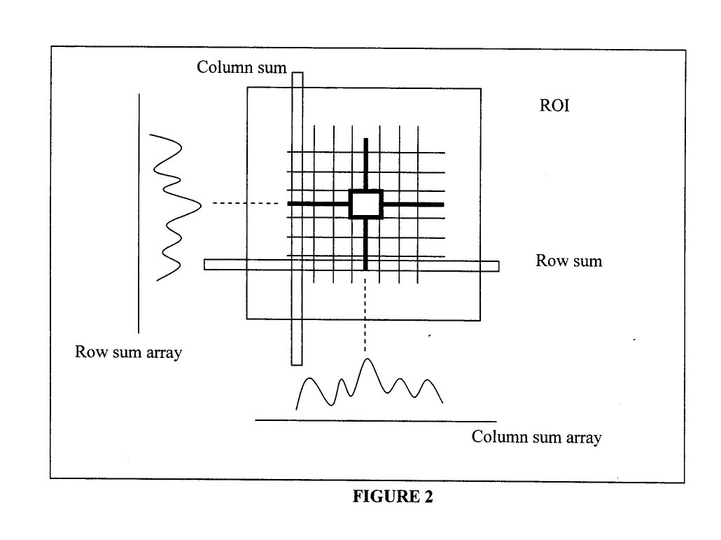

In a region of interest (ROI) in the center of the image, the present invention computes the sum of all pixels in a row and saves the sum computed for all rows, as shown in FIG. 2.

FIG. 2 illustrates the process of adding all pixels along the row inside the region of interest and saving the sum in a row sum array. The same procedure is carried out for the columns in the ROI for the column sum array. Once the row and column sums are found, the peaks are found in the arrays as the maximum values. The peak in the row sum array corresponds to the Y location of the cross center and the peak in the column sum array corresponds to the X location of the cross center.

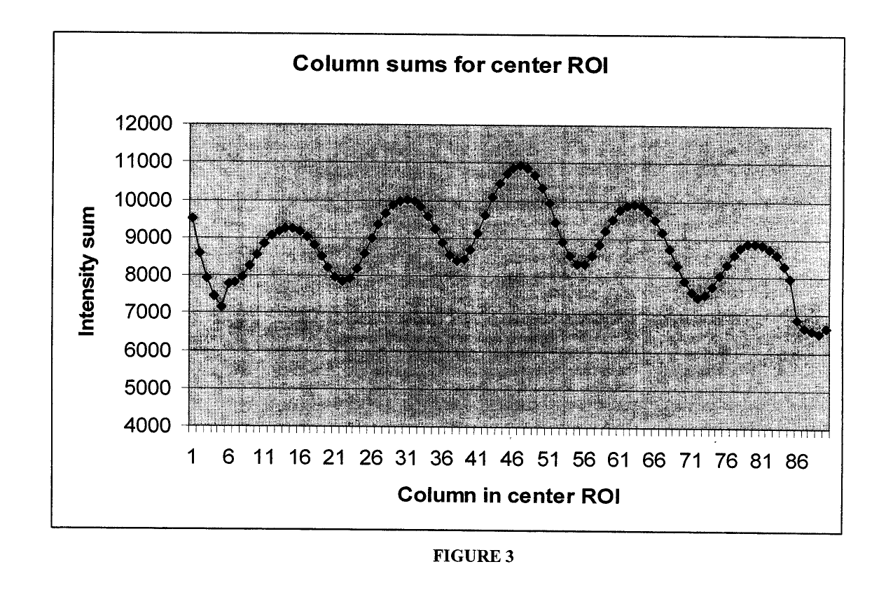

FIG. 3 is a profile of an actual column sum array in the center region of interest (ROI) for the image in FIG. 1. The profile of row sums is similar.

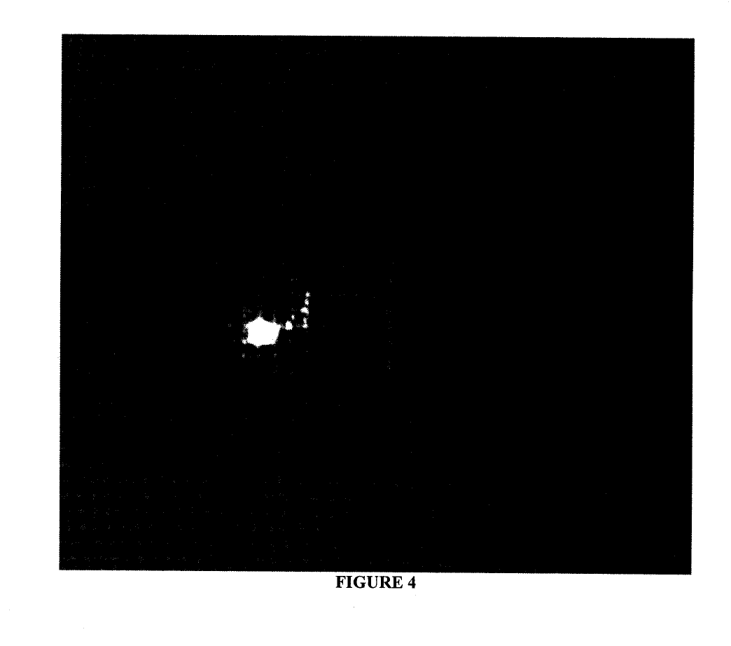

FIG. 4 illustrates the results of the center finding for the image of FIG. 1. The red square indicates the center ROI where the row and column sums are computed. The red dot in the center shows the computed cross center.

Extract Vertical and Horizontal Line Features

The next process is to extract the vertical and horizontal line features. The first step in extracting the vertical and horizontal line features is feature enhancement. To enhance the vertical and horizontal lines features the algorithm uses a simple convolution operation. The shape of the convolution kernel is illustrated below for the vertically oriented linear features. The horizontal filter is an obvious rotation of the vertical filter.

| | | |
|---|---|---|
| −1 | 0 | 1 |
| −1 | 0 | 1 |
| −1 | 0 | 1 |
| −1 | 0 | 1 |
| −1 | 0 | 1 |

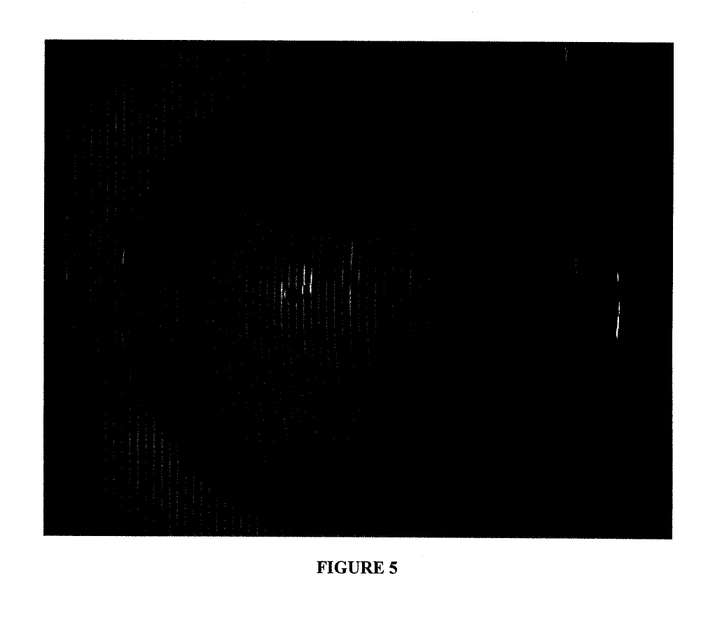

The dimension of this convolution kernel is (2w+1)×3, where w is the “half-width” of the neighborhood. Odd-lengths are used in both the height and width of this filter so as to preserve the location of the filter output (zero-phase FIR filter). For a half-width of 8, the filter size is 17×3. The output of the filter is scaled to keep the pixel values between 0 and 255. The output of the vertical enhancement filter is shown in FIG. 5.

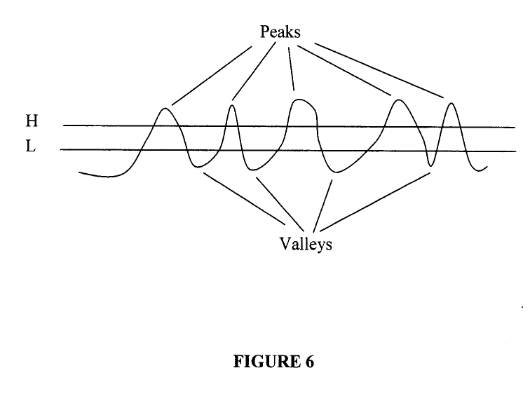

From FIG. 5 the vertical features were significantly enhanced. It was also noted that the right hand side is significantly darker (less contrast) than the left and center of the image. The next step is to scan the image to detect the peaks. The peaks will need to be adaptively determined to tolerate the non-uniform illumination present in the image. The novel peak detector strategy is explained with reference to FIG. 6.

The peak detection algorithm finds the local minima and maxima for a one dimensional profile. Two thresholds are used. A high threshold, indicated H in FIG. 6, must be exceeded for a peak to be detected and a low threshold, indicated L in FIG. 6, must be exceeded in the negative direction for a valley to be detected. To compute the high and low threshold values, the algorithm sorts all values in the vector being processed and take the 45% point and 65% point as the low and high threshold values, respectively. This separation of the thresholds (as opposed to using 50% for both thresholds) provides a certain amount of noise immunity for the local minima and maxima.

To provide adaptability of the thresholds across the image, the thresholds are computed over regions along the profile vector. The peak detection algorithm is applied to each row in the vertical feature enhancement image with four equally spaced sub-regions (where the thresholds are re-computed). To each peak the algorithm assigns the value of 255 and 0 to all other pixels. This has the effect of thinning and “binarizing” the vertical feature enhancement image.

Occasionally, certain vertical features in the original image will lead to small (one-or two pixels) features in the thinned image. To remove these noise features, a standard “shot noise” removal filter is performed. In this filter, the neighborhood around a pixel is searched. If it is surrounded by zero values, the pixel value is set to zero.

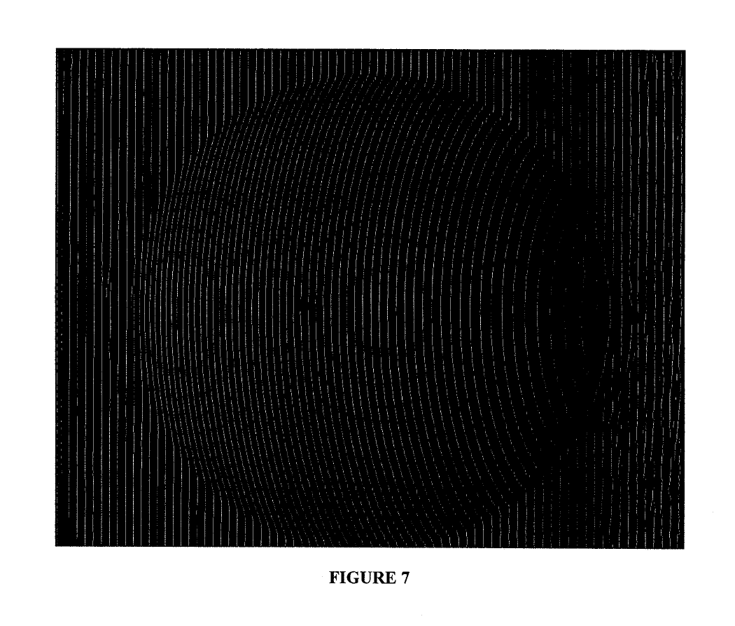

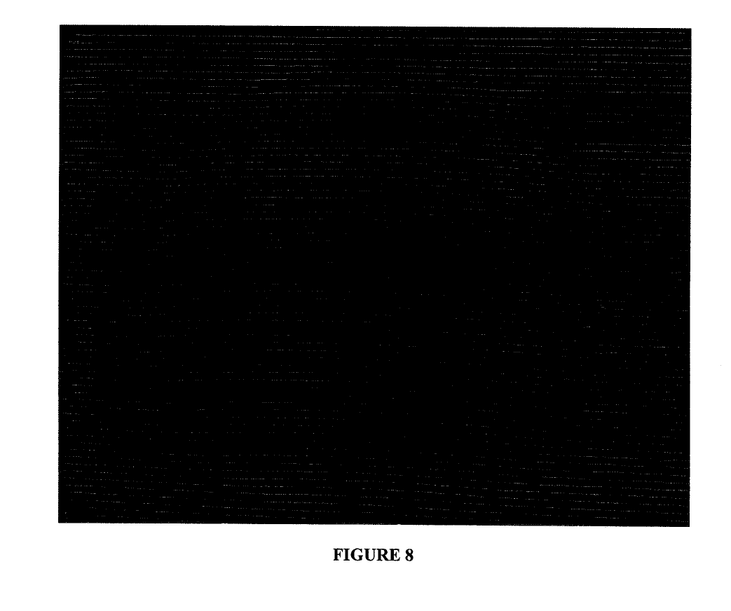

The final result of the vertical features processing is shown in FIG. 7. The processing results for the horizontal features is shown in FIG. 8. This corresponds to the image shown in FIG. 7.

Find Intersections

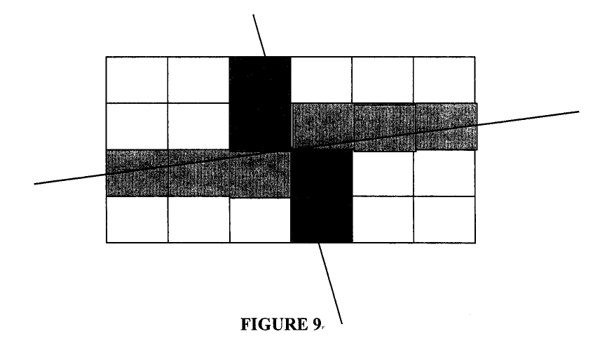

In the next step of the algorithm, the intersection of the vertical and horizontal thinned images is found. For most pixels, this is a simple matter of checking that the value of a pixel in both images is 255. However, it is possible that due to the manner in which the continuous linear features are made discrete, that a vertical and horizontal line will actually be present, but the intersection will not be detected. This can happen when the continuous horizontal linear feature has a slight positive slope, the continuous vertical linear feature has a nearly vertical negative slope, and the continuous lines just happen to cross at the boundary of a pixel. This is illustrated in FIG. 9.

In FIG. 9, the dark squares represent a discrete version of the vertical line and the light gray squares represent a discrete version of the horizontal line. Notice that the linear features actually intersect, but the discrete pixels do not have the same pixel in common indicating the intersection in the simple test. The probability of this occurring at a node is very low, but since there are over 1,000 nodes to process, it is actually likely to occur one or more times per image. Note that the situation also occurs when the continuous horizontal linear feature has a slight negative slope, the continuous vertical linear feature has a nearly vertical positive slope, and the continuous lines just happen to cross at the boundary of a pixel. To handle these situations, the line intersection test is modified as follows:

The current pixel being considered is at “00”.

Let the pixel values for a 2×2 neighborhood in the vertical thinned image be V00, V01, V10, and V11. The corresponding horizontal thinned image pixel values are H00, H01, H10, and H11.

If V11=H00=255, the lines intersect.

Or if V00=V11=H01=H10=255, the lines intersect.

Or if H00=H11=V01=V10=255, the lines intersect.

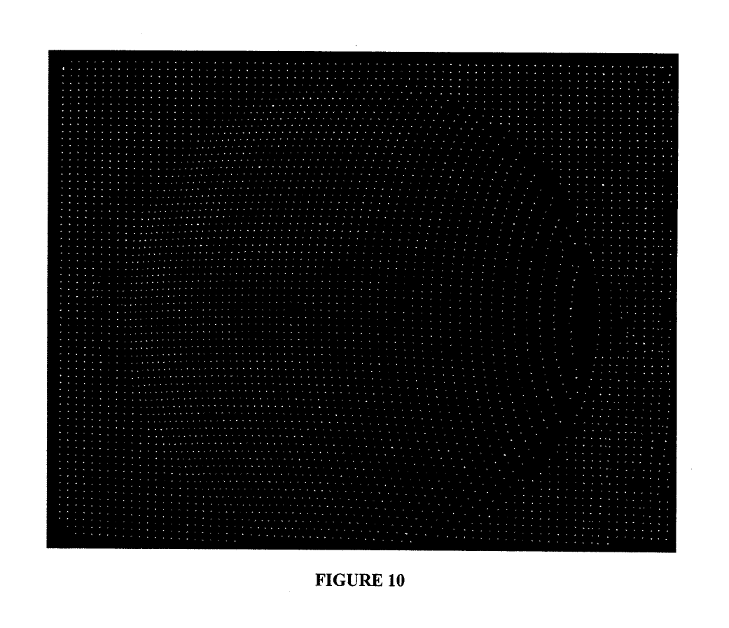

Using these digital line intersection rules, the output image from the thinned images in FIGS. 7 and 8 is shown in FIG. 10.

In the image of FIG. 10, the spots have been greatly expanded in size for inclusion in this document. In the actual image each dot was a single pixel.

Find 4-Connected Node Neighbors

After the center of the cross has been found (as shown in FIG. 4) and the linear feature intersections are located, (illustrated in FIG. 10), the next step is to begin finding the nodes. Each node contains a “Point” object. A Point object holds the x,y pixel location of the node. The nodes are stored in a two-dimensional array so that it is easy to find the neighboring nodes. The size of the node array is 101×101. The center node is located at element 50×50. The array is indexed Nodes [x,y] where x indicates the column (center is 50), and y indicates the row (center is 50). The node to the right of the center node would be stored at index (51,50). X increases to the right. The node above the center node would be stored at index (50,49). Y increases in the downward direction. At each node the algorithm stores the pixel location of the node. If the pixel location of the center node (center of the cross) is 640, 512, then Nodes [50,50]=Point (640, 512).

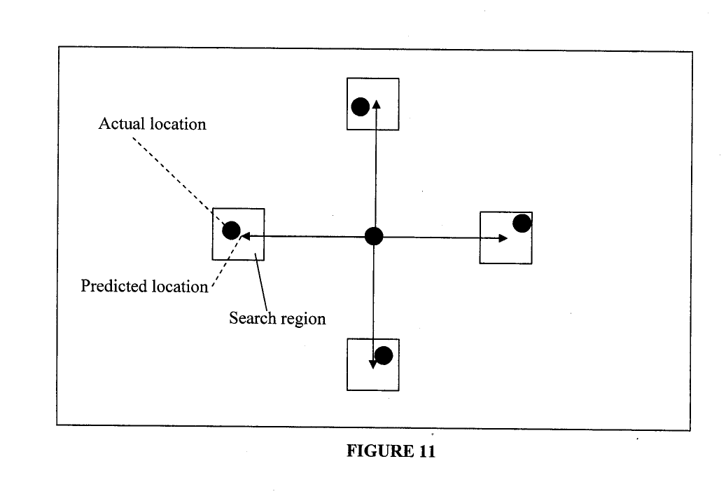

When the Nodes array is created, all node values are assigned Point (0,0). The algorithm uses this value to indicate a node that has not yet been found. If a node needs to be deleted, the instant algorithm simply overwrites its pixel location with Point (0,0). For a given node, the 4-connected neighbors are those to the right, above, left, and below. The 8-connected neighbors would include those in the diagonal directions from the center node. In the center of the image, the grid intersections are almost regularly spaced. This can be exploited by searching a fixed distance from the center node to where the neighboring node is expected to be. This is illustrated in FIG. 11.

As shown in FIG. 11, in the center, the nodes are almost regularly spaced so that the algorithm can predict about where the neighbors should be with little difficulty. The predicted location is searched looking for an intersection as shown in FIG. 10. The closest pixel found to the predicted location is used as the actual location of the neighbor.

As each direction is searched for a neighboring node, the following steps are performed.

If the node is already found in a given direction, the node is not searched again.

If the node has not yet been found (it has value 0,0), the node is searched for.

If the new neighbor node is found, its location is saved in the Nodes array. The node is also pushed onto a stack to look for the new node neighbors.

The pixel value is set in the intersection image (and the pixels in the immediate neighborhood) to zero so to prevent accidentally assigning another node to the same intersection in subsequent searches.

Initially, the center node is pushed onto the stack and then the process executes the neighborhood finder until the stack is empty.

When the stack is empty, it is almost a certainty that not all nodes would have been found (unless the image was for a flat plane). A second pass is made over the nodes where the algorithm uses the location of found node neighbors to predict where missing neighbors should be. This provides a great amount of adaptability to handle the cases where:

The nodes are close together in the x direction but far apart in the y direction as in the left side of the image in FIG. 10.

The nodes become far apart as in the right side of the image in FIG. 10.

The node paths curve as in the right side of the image in FIG. 10 and along the edges of the sphere.

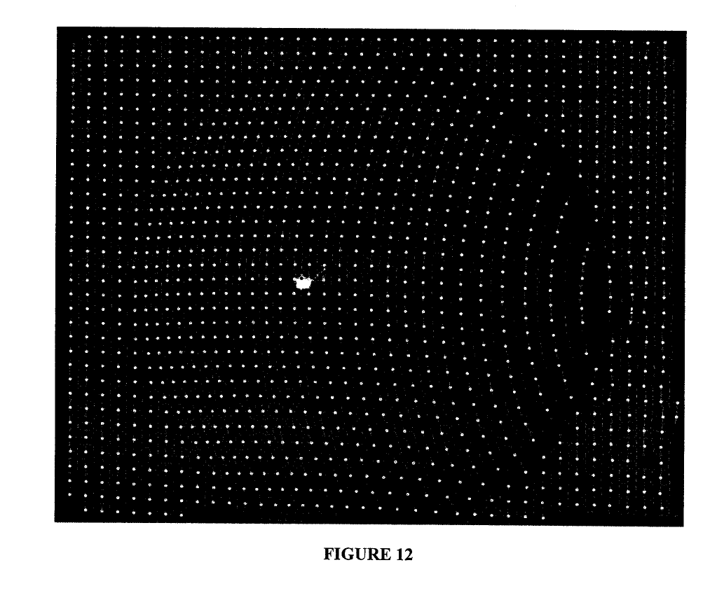

At each subsequent pass, the search window is continually increased about the predicted node location (by two pixels) until all reasonable nodes have been found. The final results are illustrated in FIG. 12.

In FIG. 12, the nodes alternate color between rows and columns to show proper topology of the nodes. A common processing error is to assign a node to the wrong row or column. The cyan connecting lines also help show the nodes were properly placed in the correct relationship to their neighbors.

While this processing algorithm is fast and reasonably robust, it is anticipated that certain image artifacts will cause errors which could propagate through the reconstruction processing to yield a surface representation that is not correct in certain areas of the image. The effects of these errors can be mitigated by the use of a post-processing step which looks for neighbors that appear to be too close, too far, or at too big an angle, with respect to neighbors. Once a neighborhood with artifacts such as these are found, the nodes could be easily deleted by setting the node value to Point (0,0). An editor could also be provided to allow manual removal of the problem nodes.

Optical/Camera Hardware System

As mentioned in the background section, the optical system was designed to shorten the overall layout. The following states the specifications.

A design goal is to have short optical tubes. To accomplish this, a telephoto lens design (positive lens followed by negative lens) was used. No vignetting of the images is allowed. A front view (at 710 nm) for focusing and pupil acquisition is provided. The image sensor is 6.6 mm×5.3 mm (8.46 mm diagonal - - - 4.23 is half diagonal) and is provided by a USB 2 camera. The grid reticle is chrome on glass 0.009 mm line width, 0.075 mm line spacing, and 19 mm in diameter. The optics are designed so that at the cornea the grid is 0.018 mm line width, 0.15 mm line spacing, 20 mm diameter. The view of the cornea is such that the coverage is 16.5 mm×13.25 mm. (21.16 mm diagonal - - - 10.58 is half diagonal). The angle between the projection and measurement arms is 12 degrees. The nominal working distance is 175 mm.

Optical Layout

The optical analysis below is performed at 550 nm. Checked front view camera at 710 nm and optimum focus is only 0.2 mm different (negligible). Inside of tubes lined with flock paper #65 to reduce stray light.

Front View

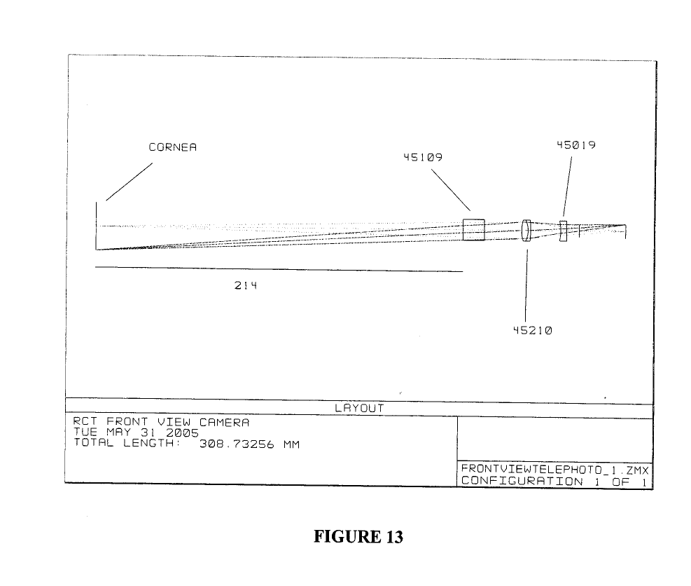

The Zemax layout for the front view camera is shown in FIG. 13. A 16.5×13.25 mm region at the corneal plane is imaged onto a 6.6×5.3 mm camera sensor via the two element telephoto lens consisting of a +35 mm lens (45210) and −48 mm focal length lens (45019). The distance from the corneal plane to the front of the right angle prism (45108) is 219 mm. This is 40 mm longer than the grid camera working distance so that the physical extent of the cameras (41 mm high, tapered) will not interfere with each other. The distance between the edge of the prism and the positive lens is 10 mm. The distance between the positive and negative lenses is 11.5 mm. The distance between the negative lens and the image plane is 59.5 mm.

The maximum diameter of the corneal region of interest is 21.16 mm.÷

$$\begin{matrix}CD = \sqrt{{16.5}^{2} + {13.25}^{2}} \\ = 21.16\end{matrix}$$

The ray tracing indicates that no vignetting occurs for up to a 10 mm aperture at the positive lens. The overall magnification of the front view camera is −0.4 (−0.3946). All four orientation combinations for the two lens telephoto lens were evaluated and the optimum configuration is indicated in FIG. 13.

Grid Camera

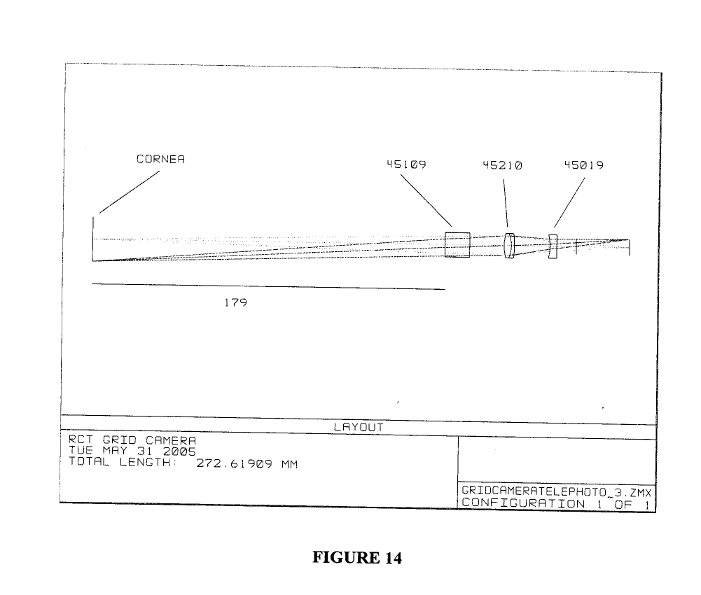

The Zemax layout for the grid camera is shown in FIG. 14. A 16.5×13.25 mm region at the corneal plane is imaged onto a 6.6×5.3 mm camera sensor via the two element telephoto lens consisting of a +35 mm lens (45210) and −48 mm focal length lens (45019). The distance from the corneal plane to the front of the right angle prism (45108) is 179 mm. The distance between the edge of the prism and the positive lens is 10 mm. The distance between the positive and negative lens is 17.5 mm. The distance between the negative lens and the image plane is 39.74 mm.

As before, the maximum diameter of the corneal region of interest is 21.16 mm.÷

$$\begin{matrix}CD = \sqrt{{16.5}^{2} + {13.25}^{2}} \\ = 21.16\end{matrix}$$

The ray tracing indicates that no vignetting occurs for up to a 10 mm aperture at the positive lens. The overall magnification of the grid view camera is −0.4 (−0.4041).

Grid Projection

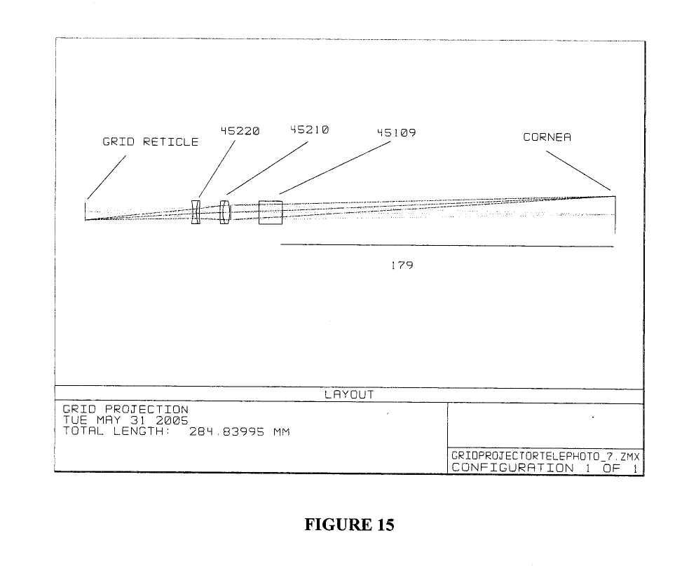

The Zemax ray tracing for the grid projection is shown in FIG. 15. A 10-mm diameter region of the grid reticle imaged onto a 20-mm diameter (plus projection distortion) via the telephoto lens consisting of a −48 mm (45019) lens and a +35 (45210) mm lens. The distance from the grid to the negative lens is 62 mm. The distance between the lenses is 10.5 mm. The distance between the positive lens and the prism is 10 mm. The distance from the prism to the corneal plane is 179 mm which is the same as for the grid camera. The overall magnification of the grid projection is −2.0 (−2.06).

Grid Illumination

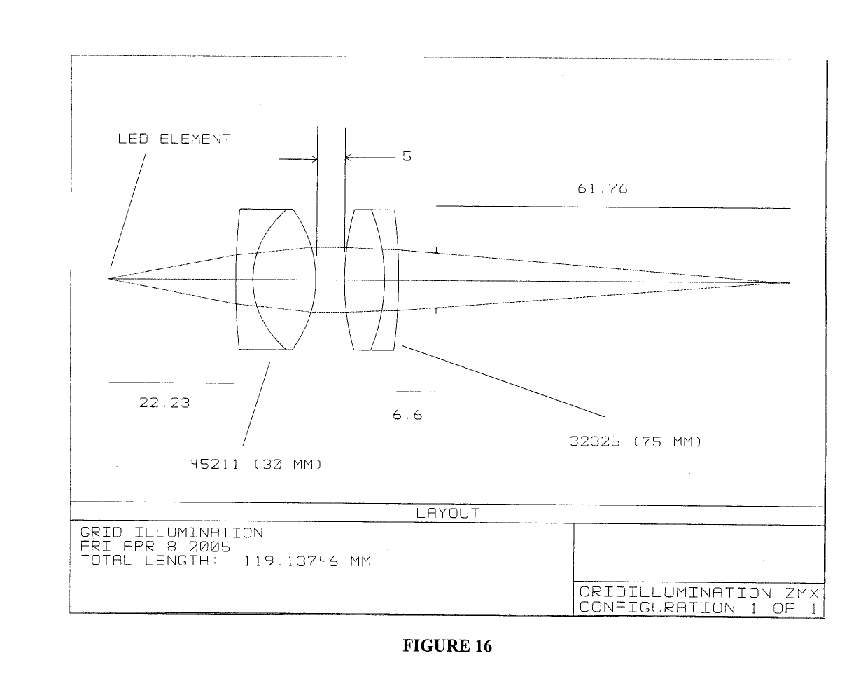

The grid illumination system consists of a 1W Luxeon LED with its integral lens ground off. The lens must be ground off so that the lens system can provide a uniform illumination pattern at the grid reticle. The LED requires mechanical mounting (#4×40 socket head works well) to an aluminum heat sink. A suitable heat sink is the AAVID Thermalloy 1.83×1.83×0.5 inch, no holes—part number 568000B00000 (digikey part number HS291-ND). The LED may be directly attached to the heat sink as the bottom of the LED package is electrically isolated from the contacts on top of the package. The LED is imaged onto the plane of the first grid projector lens via a 30 mm collimation lens (45211) and a 75-mm focusing lens (32325). The optical path from the LED to the grid reticle and the first lens of the grid projector is illustrated in FIG. 16.

Note that the grid reticle is 6.6 mm from the positive lens. If the back surface of the lens and the grid reticle are at the same location any dust on the focusing lens will be projected at the cornea. The distance from the LED element to the collimation lens is 22.23 mm. The distance between the lenses is 5 mm and is not critical. The distance from the focusing lens to the first grid projection lens is 68.37 mm.

All orientation configurations of the collimation lens and the focusing lens were evaluated and the orientation shown in FIG. 16 provides the minimum aberrations.

Optomechanical

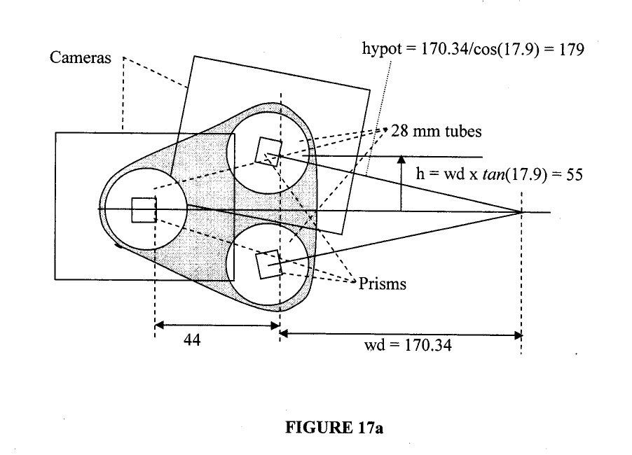

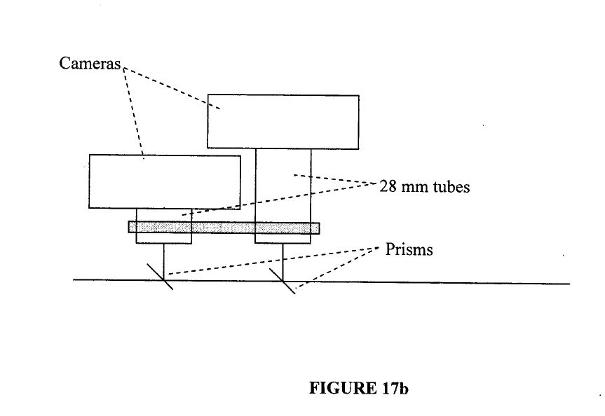

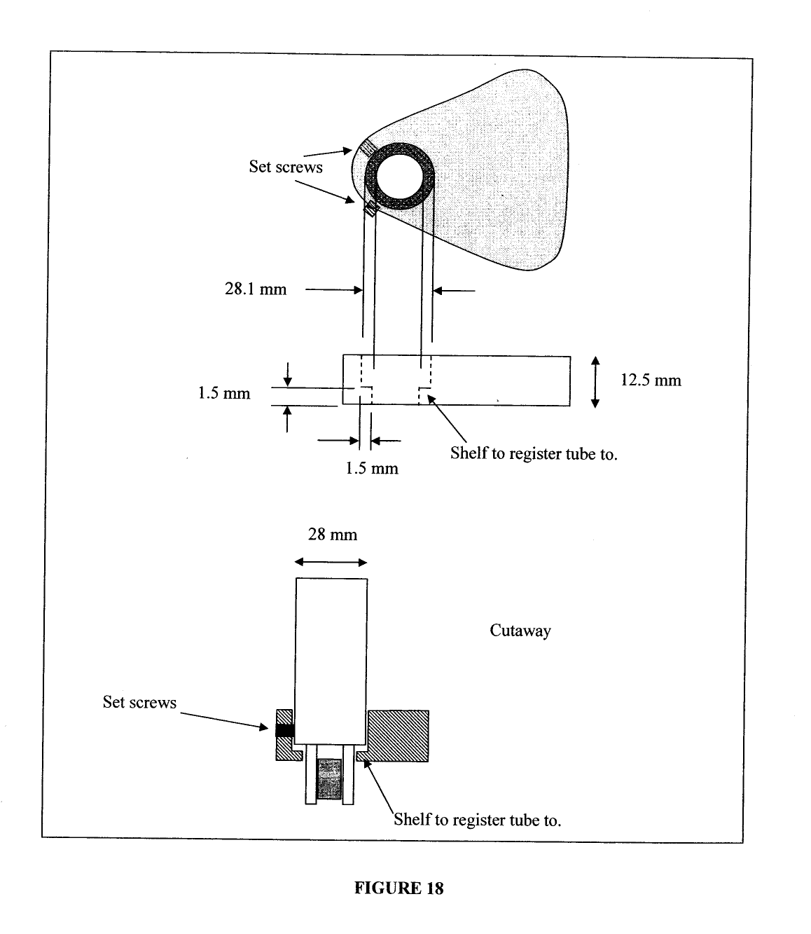

The optomechanical arrangement top and side views are illustrated in FIGS. 17 and 18, respectively. FIG. 17 also shows the distance h from the system optical axis to the center of the grid camera tube given the angle between the grid projector and grid camera is 35.8 degrees and the working distance is 170 mm (from the plane containing the prisms). From the top view, the extent to which the cameras overlap each other is shown, but from the side view it can be seen that the front view camera is 40 mm lower than the grid view camera. The lenses and apertures are enclosed in 30 mm diameter tubes. The prisms are contained in an enclosure similar to the PAR design. The gray region in the top and side view is one possibility for providing structure for the three tubes. Two of these triangular shapes can be aligned with standoffs to form a triangular cage. Set screws in the corners hold the optics tubes.

When not required, the front view camera and optics tube is not integrated. The distance between the center of the holes for the grid camera and grid projector is 110 mm.

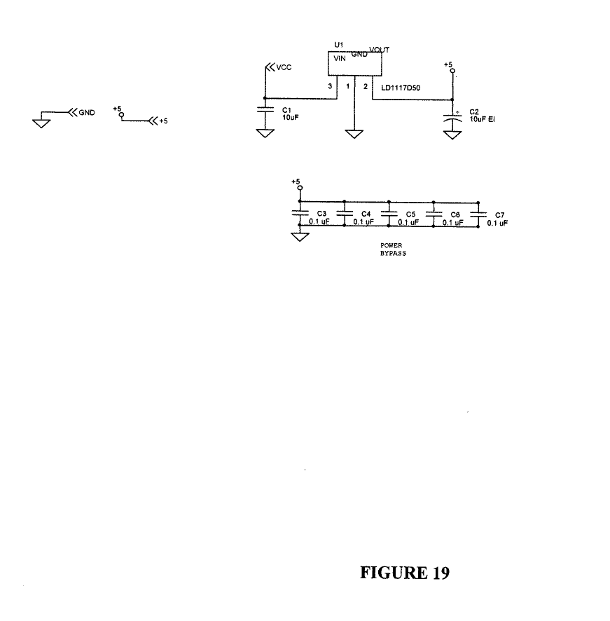

Mounting detail is shown in FIG. 19. The optical parts are to be primarily assembled from off the shelf components the tubes are 28 mm OD and 25 mm ID. The prism will be held in place by a “Clevis mount” at the end of the optical tube. The prism will be fixed relative to the camera in the optical tube. The rotation of the prism to be aimed for the 17.8 deg angle will be made by inserting the tube into the triangular frame, twisting the tube to the correct alignment and fixing the position with two set screws as illustrated in FIG. 19.

Flash Controller Electronics

In this section the design of the flash controller electronics is described.

The flash controller printed circuit board (PCB) performs functions related to the illumination and digital input/output processing of the system. Specifically, the board turns light emitting diodes (LED) on and off, illuminate an LED at multiple intensities and processes digital input/output for switches, indicator lights, etc.

Specific requirements for the flash controller PCB are as follows:

4 TTL compatible inputs from host PC (GPO).

4 TTL compatible outputs to host PC. (GPI).

4 TTL compatible inputs for external events.

2-wire serial interface to potentiometer.

1 flash enable line, on/off toggle.

1 chip select output for potentiometer.

1 clock input for potentiometer serial interface.

8-wire JTAG port for programming the PLD chip.

Inputs to the controller PCB are TTL-compatible digital inputs consisting of 1 3-wire serial input: The 3-wire serial input is organized as 1 data clock, 1 data input and 1 data latch (indicates writing of data complete and command parsing should begin).

The controller PCB commands are issued as 24 serial bits divided into 3 8-byte words. The first 8 bits define the command issued to the interface board. The second 8 bits constitute data byte 1 (potentiometer command byte) and the third 8 bits constituted data byte 2 (potentiometer data byte). After the 24 bits are written, a low to high transition of the data latch parses data in interface command byte. I.e. the specified command immediately executes.

The following are the valid commands to be issued to the controller PCB:

Interface Command #1, 0x07—Reset board, data bytes ignored.

Interface Command #2, 0x01—data bytes 1 and 2 to potentiometer.

Interface Command #3, 0x02—End Potentiometer programming, data bytes ignored.

Interface Command #4, 0x04—Set Flash to on, data bytes ignored.

Interface Command #5 0x05—Set Flash to off, data bytes ignored.

Interface Command #6, 0x06—Clear data input latch, data bytes ignored.

Interface Command #7, 0xff—Test Mode, data bytes ignored.

Data byte 1 defines the command to program the light intensity potentiometers on the flash controller PCB. Data in this byte is sent to board in reverse order (MSB first). Data is sent in the following format (X=Ignore):

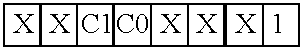

| C1 | C0 | Command | Summary |
|---|---|---|---|
| 0 | 0 | None | No command executed |
| 0 | 1 | Write | Write data in data byte to |
| | | | potentiometer |
| 1 | 0 | Shutdown | Potentiometer enters shutdown |
| | | | mode (exits shutdown mode when |
| | | | new data byte is written) |

Data byte 2 is the position of the potentiometer wiper and thus the intensity of the LED. Data is sent in reverse order, MSB first and is valid over the range of decimal 0 to 255. Data is sent in the following format:

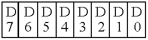

Once data has been sent and after a 75 ms delay, follow with an Interface Command #3 (0x02) to signal the board that potentiometer programming is finished.

The actual light level may be calculated using the following data byte to light level conversion where decimal 200=2.18V (Lowest discernable light level) and decimal 0=3.8V (Maximum light intensity). Using an approximate 1^{st} order regression, Voltage (DC Volts)=potentiometer position # (in decimal {range 0 to 255})*−0.00875+4.018.

The flash controller PCB is also responsible for digital input and output control. The controller PCB contains a 4-wire parallel input to controller for external events. The “acquire” button of the topographer is latched to GPI(0) until a specific command (Interface Command #6) is issued. Data is removed from the latch upon writing interface command #6. If a second external event occurs while data is latched, the event is ignored. The remaining 3 inputs (GPI(1) through GPI(3)) are not latched, data is passed through the PLD to appropriate GPI lines.

The controller PCB also contains a test mode function to verify the proper operating condition of the PCB. Upon entering into the test mode, the input data latches will transition to 0x0f (all 1's). The test mode is cleared by writing of interface command #7 (Reset), however GPI(0) is not cleared with reset, a command #6 must be issued to clear it. A reset mode is also available that deselects all chips, turns the flash off (if on) and sets all test outputs to a high impedance state.

The flash duration is set in hardware to 200 ms. To generate a 200 ms flash, immediately issue a flash on Command #4 followed immediately by a flash off Command #5. For torch mode (continuous illumination) issue only a flash on Command #4 and only issue a flash off Command #5 when ready to extinguish the light.

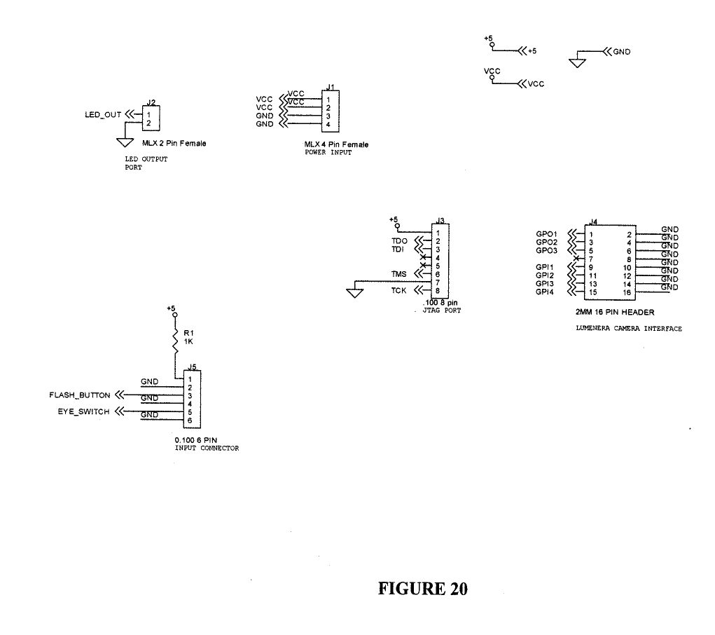

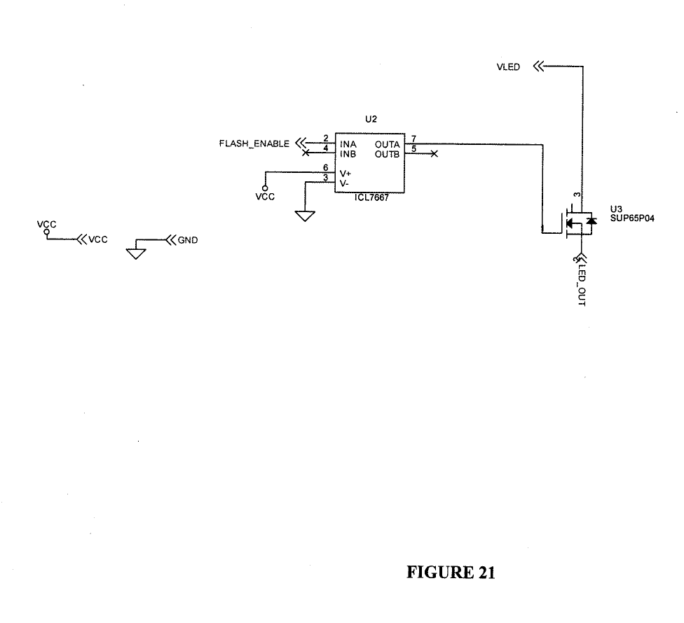

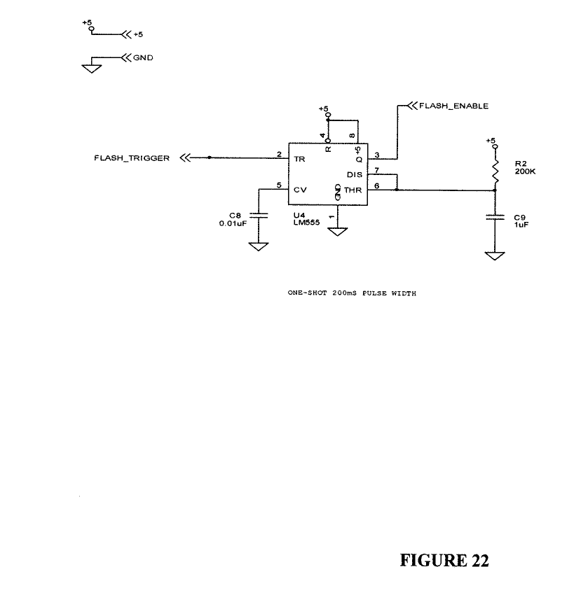

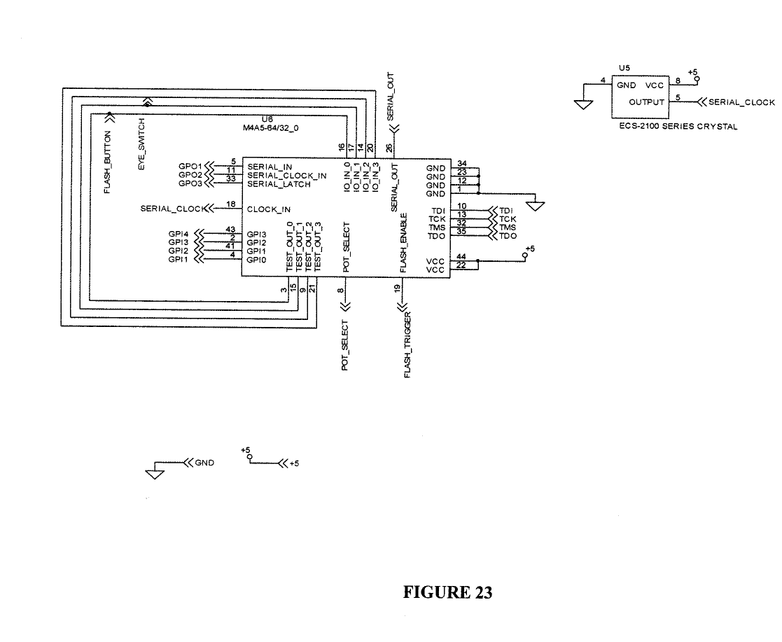

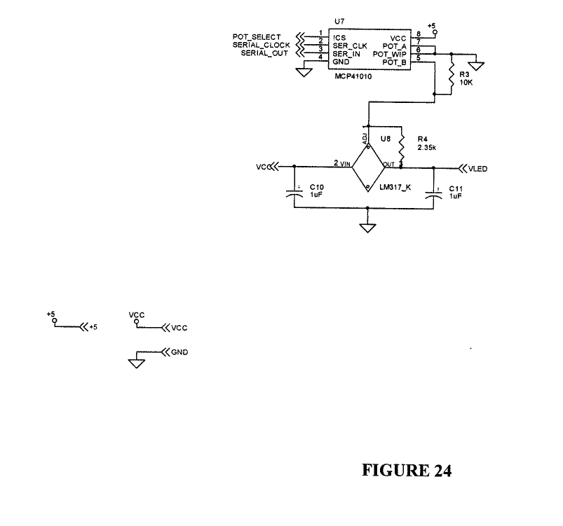

The schematics for the voltage power supply (FIG. 20); connectors and switches (FIG. 21); the LED array drivers (FIG. 22); the one shot trigger (FIG. 23); the PLD (FIG. 24); and the programmable 2 AMP current power supply (FIG. 25) are shown.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of determining corneal topography comprising the steps of:
a.) staining the tears of an eye;
b.) projecting a grid arranged in vertical and horizontal lines onto said stained tears, said grid having a sufficient size to extend at least across a cornea of an eye;
c.) capturing an image of said projected grid with a camera;
d.) transferring said image to a computer for processing;
e.) determining the center of said cornea by counting the rectangles formed by said vertical and said horizontal lines extending across said cornea;
f.) locating the intersections of said vertical and said horizontal lines;
g.) assigning an X and a Y coordinate to each said intersection.

2. The method of determining corneal topography of claim 1 wherein said method includes the step of enhancing said vertical and said horizontal lines with a convolution operation prior to locating said intersections.

3. The method of determining corneal topography of claim 2 wherein said enhancement of said vertical and said horizontal lines includes thinning of said vertical and said horizontal lines.

4. The method of determining corneal topography of claim 1 including the step of post processing, whereby intersection locations that are too close, too far or at too big of an angle with respect to adjacent intersections are deleted.

5. The method of determining corneal topography of claim 1 including an editor constructed and arranged for manual removal of intersection locations that are too close, too far or at too big of an angle with respect to adjacent intersections.
